(19) United States
(12) Patent Application Publication
(10) Pub. No.: US 2025/0361989 A1
(43) Pub. Date: Nov. 27, 2025

(54) METHOD AND INTERNET OF THINGS (IOT) SYSTEM FOR SAFELY REPLACING A GAS PIPELINE NETWORK COMPONENT BASED ON A SUPERVISION NETWORK

(71) Applicant: CHENGDU QINCHUAN IOT TECHNOLOGY CO., LTD., Chengdu (CN)

(72) Inventors: Zehua SHAO, Chengdu (CN); Yaqiang QUAN, Chengdu (CN); Xueqiang LEI, Chengdu (CN); Yuefei WU, Chengdu (CN)

(73) Assignee: CHENGDU QINCHUAN IOT TECHNOLOGY CO., LTD., Chengdu (CN)

(21) Appl. No.: 19/291,636

(22) Filed: Aug. 6, 2025

(30) Foreign Application Priority Data
Jul. 21, 2025 (CN) 202510998219.3

(51) Int. Cl. F17D 3/01; G05B 19/4063

(52) U.S. Cl. CPC F17D 3/01; G05B 19/4063; G05B 2219/33034; G05B 2219/41108; G05B 2219/45233

(57) ABSTRACT

A method and an Internet of Things (IoT) system for safely replacing a gas pipeline network component are provided. The method includes: acquiring a component feature of a gas numerical control component based on a data center; determining a replacement necessity degree of the gas numerical control component based on the component feature; determining a replacement parameter based on the replacement necessity degree; generating a replacement task instruction and a gas shutoff parameter based on the replacement parameter; in response to receiving a shutoff parameter confirmation instruction, generating a gas shutoff instruction and transmitting the gas shutoff instruction to a gas supply control device; generating gas shutoff reminder information; and in response to receiving a replacement completion message, determining a replaced component and updating a data partition corresponding to the replaced component.

### CROSS-REFERENCE TO RELATED APPLICATIONS

This application claims the priority of Chinese Patent Application No. 202510998219.3, filed on Jul. 21, 2025, the contents of which are hereby incorporated by reference.

### TECHNICAL FIELD

The present disclosure relates to the field of pipeline network component replacement, and particularly to a method and an Internet of Things (IoT) system for safely replacing a gas pipeline network component based on a supervision network.

### BACKGROUND

A gas pipeline network, as a key component of urban energy supply, is deployed with a plurality of gas numerical control components for gas monitoring. During the long-term operation of the gas pipeline network, various factors can cause faults or performance degradation of at least one gas numerical control component among the gas numerical control components. Therefore, timely replacement of the at least one gas numerical control component is crucial for maintaining the safe operation of the pipeline network. Currently, the replacement of the at least one gas numerical control component often relies on periodic inspections or determining that the at least one gas numerical control component has completely failed, making it difficult to ensure the timeliness and accuracy of the replacement.

Therefore, it is necessary to provide a method and an Internet of Things (IoT) system for safely replacing a gas pipeline network component based on a supervision network, which can determine the replacement necessity degree of each gas numerical control component, accurately determine the at least one gas numerical control component that needs to be replaced, replace the at least one gas numerical control component, and ensure the normal operation of the gas pipeline network.

### SUMMARY

The present disclosure provides a method for safely replacing a gas pipeline network component based on a supervision network. The method is executed by a gas company management platform in an Internet of Things (IoT) system for safely replacing a gas pipeline network component based on a supervision network. The method includes: acquiring a component feature of a gas numerical control component based on a data center of the gas company management platform, the component feature comprising at least one of a data processing feature, a working feature, and an environmental feature, the component feature being acquired by a smart gas equipment object platform, uploaded to the gas company management platform via a gas company sensing network platform, and stored in a data partition of the data center corresponding to the gas numerical control component; determining a replacement necessity degree of the gas numerical control component based on the component feature; determining a replacement parameter based on the replacement necessity degree; generating a replacement task instruction and a gas shutoff parameter based on the replacement parameter, transmitting the replacement task instruction to a maintenance user sub-platform via a maintenance service sub-platform, and transmitting the gas shutoff parameter to a government safety supervision management platform via a government safety supervision sensing network platform; in response to receiving a shutoff parameter confirmation instruction from the government safety supervision management platform, generating a gas shutoff instruction, and transmitting the gas shutoff instruction to a gas supply control device via the gas company sensing network platform to control the open/closed state of the gas supply control device; generating gas shutoff reminder information and transmitting the gas shutoff reminder information to a gas user sub-platform and a citizen user platform; and in response to receiving a replacement completion message issued by the maintenance user sub-platform, determining a replaced component and updating the data partition corresponding to the replaced component.

The present disclosure also provides an Internet of Things (IoT) system for safely replacing a gas pipeline network component based on a supervision network. The IoT system includes a government safety supervision service platform, a government safety supervision management platform, a government safety supervision sensing network platform, a government safety supervision object platform, a gas company sensing network platform, a smart gas equipment object platform, a citizen user platform, a gas user platform, and a gas user service platform. The government safety supervision object platform includes a gas company management platform. The smart gas equipment object platform includes a gas supply control device. The gas user platform includes a maintenance user sub-platform and a gas user sub-platform. The gas user service platform includes a maintenance service sub-platform. The gas company management platform is configured to execute the method for safely replacing a gas pipeline network component based on a supervision network.

The present disclosure also a computer-readable storage medium. The storage medium stores computer instructions. When a computer reads the computer instructions in the storage medium, the computer executes the method for safely replacing the gas pipeline network component based on the supervision network.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary platform structure of an Internet of Things (IoT) system for safely replacing a gas pipeline network component based on a supervision network according to some embodiments of the present disclosure;

FIG. 2 is a flowchart illustrating an exemplary process of a method for safely replacing a gas pipeline network component based on a supervision network according to some embodiments of the present disclosure;

FIG. 3 is a flowchart illustrating an exemplary process for determining a replacement necessity degree according to some embodiments of the present disclosure;

FIG. 4 is an exemplary schematic diagram of an evaluation model according to some embodiments of the present disclosure; and

FIG. 5 is a flowchart illustrating an exemplary process for determining a replacement parameter of a gas region according to some embodiments of the present disclosure.

### DETAILED DESCRIPTION

The accompanying drawings, which are required to be used in the description of the embodiments, are briefly described below. The accompanying drawings do not represent the entirety of the embodiments.

When describing operations performed step-by-step in the embodiments of the present disclosure, unless otherwise specified, the order of the operations may be adjusted, some operations may be omitted, and additional operations may be included in the processes.

The embodiments in the present disclosure are for illustration and description only and do not limit the scope of application of the present disclosure. For those skilled in the art, various modifications and changes that may be made under the guidance of the present disclosure are still within the scope of the present disclosure. In addition, certain features, structures, or characteristics in one or more embodiments of the present disclosure may be appropriately combined.

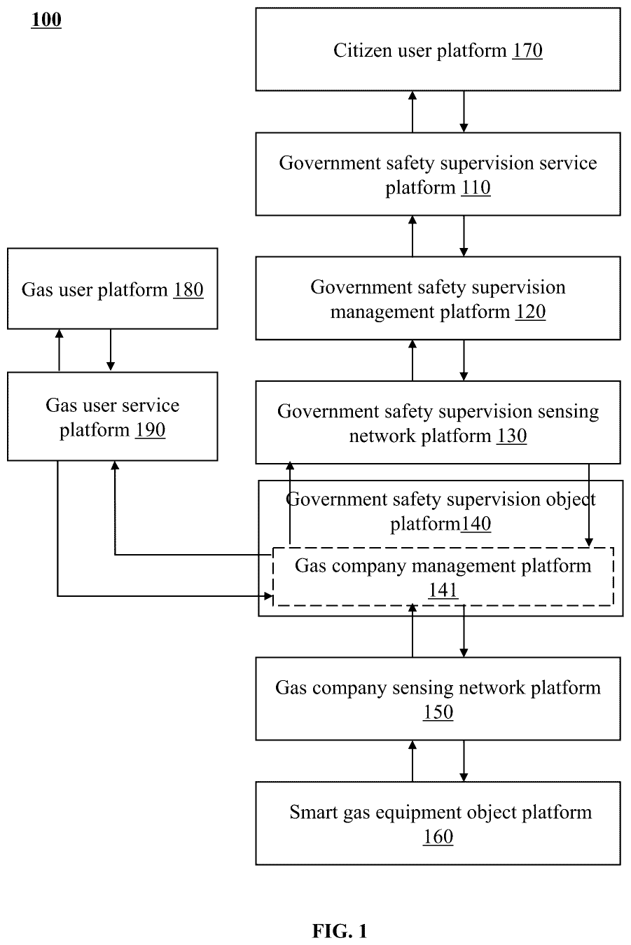

FIG. 1 is a schematic diagram illustrating an exemplary platform structure of an Internet of Things (IoT) system for safely replacing a gas pipeline network component based on a supervision network according to some embodiments of the present disclosure.

In some embodiments, as shown in FIG. 1, an Internet of Things (IoT) system 100 for safely replacing a gas pipeline network component (i.e., a target component of a gas pipeline network) based on a supervision network may include a government safety supervision service platform 110, a government safety supervision management platform 120, a government safety supervision sensing network platform 130, a government safety supervision object platform 140, a gas company sensing network platform 150, a smart gas equipment object platform 160, a citizen user platform 170, a gas user platform 180, and a gas user service platform 190.

The government safety supervision service platform 110 refers to a platform that provides safety supervision services for the government. In some embodiments, the government safety supervision service platform 110 may be configured as a server, which may perform data interaction with the government safety supervision management platform 120 and the citizen user platform 170. For example, the citizen user platform 170 may acquire gas shutoff reminder information issued by the government safety supervision object platform 140 through the government safety supervision service platform 110.

The government safety supervision management platform 120 refers to a platform for supervising and managing the safety of the gas pipeline network. In some embodiments, the government safety supervision management platform 120 may be configured as the server.

In some embodiments, the government safety supervision management platform 120 may perform data interaction upward with the government safety supervision service platform 110, and downward with the government safety supervision object platform 140 through the government safety supervision sensing network platform 130.

The government safety supervision sensing network platform 130 refers to a functional platform for managing the security of sensing communication for the government. In some embodiments, the government safety supervision sensing network platform 130 may be configured as communication devices, gateways, etc.

In some embodiments, the government safety supervision sensing network platform 130 may be used for communication between the government safety supervision management platform 120 and the government safety supervision object platform 140. For example, the government safety supervision management platform 120 may send a shutoff parameter confirmation instruction to a gas company management platform 141 of the government safety supervision object platform 140 through the government safety supervision sensing network platform 130. As another example, the gas company management platform 141 of the government safety supervision object platform 140 may send a gas shutoff parameter to the government safety supervision management platform 120 through the government safety supervision sensing network platform 130.

The government safety supervision object platform 140 refers to an object platform for generating sensing information and executing control information. In some embodiments, the government safety supervision object platform 140 may include a gas company management platform 141.

The gas company management platform 141 refers to a comprehensive management platform for information related to a gas company. In some embodiments, the gas company management platform 141 may be configured as a server and a storage device.

In some embodiments, the gas company management platform 141 may interact with the government safety supervision management platform 120 through the government safety supervision sensing network platform 130, interact with the smart gas equipment object platform 160 through the gas company sensing network platform 150, and interact with the gas user platform 180 through the gas user service platform 190.

In some embodiments, the gas company management platform 141 may be configured to execute the method for safely replacing the gas pipeline network component based on the supervision network. In some embodiments, the gas company management platform 141 may further include a processor. The processor may process data and/or information acquired from other platforms. The processor may execute program instructions based on the data, the information, and/or processing results to perform one or more functions described in this application. In some embodiments, the processor may include one or more sub-processing devices (e.g., a single-core processing device or a multi-core multi-chip processing device). Merely by way of example, the processor may include a central processing unit (CPU), an application-specific integrated circuit (ASIC), etc., or any combination thereof.

The gas company sensing network platform 150 refers to a comprehensive management platform for sensing information of the gas company. In some embodiments, the gas company sensing network platform 150 may be configured as a communication network, gateways, etc.

In some embodiments, the gas company sensing network platform 150 may be used for communication between the government safety supervision object platform 140 and the smart gas equipment object platform 160.

The smart gas equipment object platform 160 refers to a functional platform for real-time monitoring and intelligent regulation of the gas pipeline network. In some embodiments, the smart gas equipment object platform 160 may include a gas numerical control component and a gas supply control device.

The gas numerical control component refers to a device that uses numerical control technology to control and monitor gas. In some embodiments, the gas numerical control component may include a gas flow sensor, a gas pressure sensor, and a gas temperature sensor. In some embodiments, the gas numerical control component may be deployed inside pipelines of a plurality of gas pipelines of the gas pipeline network for monitoring the gas condition inside the gas pipelines.

It should be noted that the gas numerical control component in the present disclosure is a battery-powered device, meaning that the gas numerical control component has a battery installed internally.

The gas supply control device refers to related equipment for regulating gas supply. In some embodiments, the gas supply control device may be deployed inside pipelines of a plurality of gas pipelines of the gas pipeline network or at pipeline entrances/exits and other locations, for regulating and controlling gas flow, gas pressure, gas delivery direction, etc. For example, the gas supply control device may be a gas valve, etc.

In some embodiments, the smart gas equipment object platform 160 may interact with the gas company management platform 141 through the gas company sensing network platform 150.

The citizen user platform 170 refers to a platform for interacting with citizens. In some embodiments, the citizen user platform 170 may be configured as terminal devices of public media, such as television media, targeting all citizens.

In some embodiments, the citizen user platform 170 may interact with the government safety supervision service platform 110.

The gas user platform 180 refers to a platform for interacting with gas users. In some embodiments, the gas user platform 180 may be configured as terminal devices of the gas users, such as mobile phones, computers, etc., of the gas users.

In some embodiments, the gas user platform 180 may include a maintenance user sub-platform and a gas user sub-platform.

The maintenance user sub-platform refers to a functional platform that provides maintenance services and user support. In some embodiments, the maintenance user sub-platform may be configured as work terminals used by gas maintenance personnel.

The gas user sub-platform refers to a sub-platform for interacting with the gas users. In some embodiments, the gas user sub-platform may be configured as terminal devices used by the gas users.

The gas user service platform 190 refers to a platform for receiving and transmitting gas user-related data and/or information. In some embodiments, the gas user service platform 190 may be configured as a server.

In some embodiments, the gas user service platform 190 may include a maintenance service sub-platform.

The maintenance service sub-platform may refer to a platform for receiving and transmitting maintenance information.

In some embodiments, the gas user platform 180 may interact with the gas company management platform 141 through the gas user service platform 190. For example, the maintenance service sub-platform may receive a replacement task instruction from the gas company management platform 141 and send it to the maintenance user sub-platform. As another example, the gas company management platform 141 sends the gas shutoff reminder information to the gas user sub-platform through the maintenance service sub-platform.

In some embodiments of the present disclosure, the IoT system for safely replacing the gas pipeline network component based on the supervision network can form an information operation closed loop among various functional platforms, operate coordinately and regularly under the unified management of the gas company management platform, and achieve informatization and intelligence in the safely replacement of gas pipeline network components.

It should be noted that the above description of the IoT system for safely replacing the gas pipeline network component based on the supervision network and its platforms is for convenience of description only and may not limit the present disclosure to the scope of the examples provided. It is understood that for those skilled in the art, after understanding the principle of the system, they may make any combination of the various platforms or form subsystems connected to other platforms without departing from the principle.

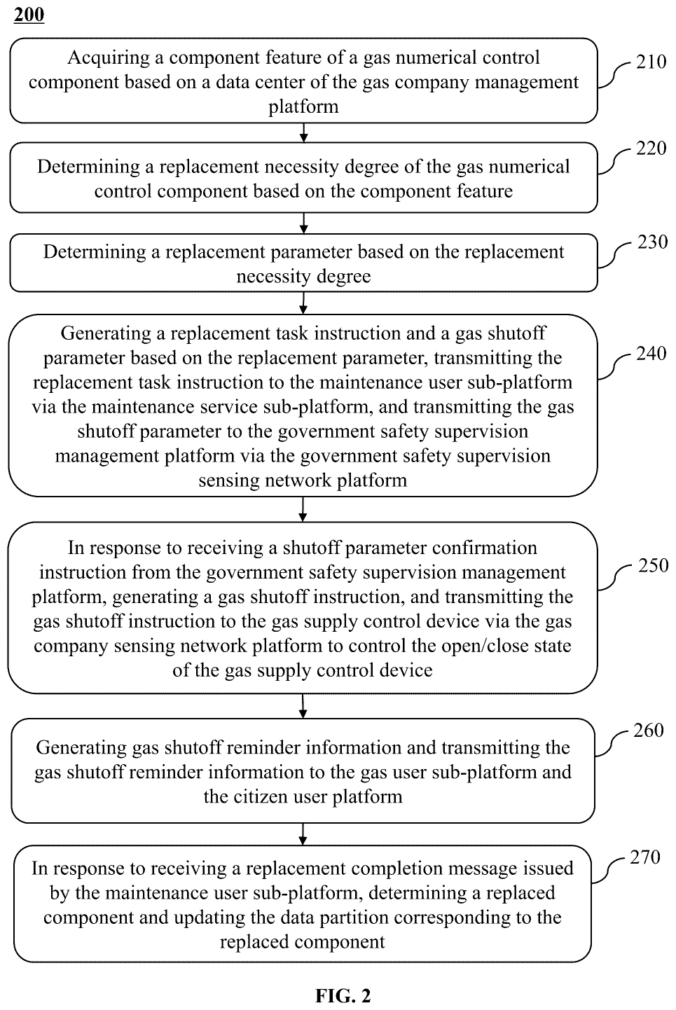

FIG. 2 is a flowchart illustrating an exemplary process 200 for safely replacing a gas pipeline network component based on a supervision network according to some embodiments of the present disclosure.

In some embodiments, the process 200 may be implemented by the IoT system 100, and may be executed by the gas company management platform 141 in the IoT system 100. For example, the process 200 may be executed by a processor in the gas company management platform 141.

As shown in FIG. 2, the process 200 includes the following operations 210-270.

In 210, a component feature of a gas numerical control component is acquired based on a data center of the gas company management platform.

The data center refers to a module for centralized storage and processing of data. In some embodiments, the data center may include a plurality of data partitions.

The data partition refers to a sub-area in the data center for storing data. In some embodiments, each gas numerical control component has its corresponding data partition, and the data partition of the data center corresponding to the gas numerical control component may be set by default by the processor or pre-divided and set by technical personnel of the data center.

The component feature refers to a relevant feature of the gas numerical control component. In some embodiments, the component feature may include at least one of a data processing feature, a working feature, or an environmental feature.

The data processing feature refers to a relevant feature of the gas numerical control component that processes data. For example, the data processing feature may include a data collection parameter and a data transmission parameter.

The data collection parameter refers to a parameter for the gas numerical control component collecting data, such as a data collection frequency and a data collection amount. The data collection frequency refers to a count of times the gas numerical control component collects data per unit time. The data collection amount refers to an amount of data collected by the gas numerical control component in a single collection.

The data transmission parameter refers to a parameter for the gas numerical control component transmitting data, such as a data transmission frequency and a data transmission amount. The data transmission frequency refers to a count of times the gas numerical control component transmits data per unit time. The data transmission amount refers to an amount of data transmitted by the gas numerical control component in a single transmission.

The working feature refers to a relevant feature of the operation of the gas numerical control component. For example, the working feature may include a start-stop frequency and a working duration.

The start-stop frequency refers to a total count of times the gas numerical control component is started or stopped. The working duration refers to a length of time the gas numerical control component works continuously after a single start.

The environmental feature refers to a relevant feature of an environmental condition where the gas numerical control component is located. For example, the environmental feature may include an environmental temperature, an environmental humidity, an environmental pH value, etc., of the environment where the gas numerical control component is located.

In some embodiments, the component feature of the gas numerical control component may be acquired by the smart gas equipment object platform, uploaded to the gas company management platform via the gas company sensing network platform, and stored in the data partition of the data center corresponding to the gas numerical control component.

The smart gas equipment object platform may acquire the data processing feature and the working feature of the gas numerical control component through calculation based on data uploaded by the gas numerical control component, and acquire the environmental feature of the gas numerical control component based on other monitoring devices (e.g., temperature sensors deployed around the gas numerical control component).

In some embodiments, the processor may directly retrieve the component feature of the gas numerical control component from the data center of the gas company management platform.

In 220, a replacement necessity degree of the gas numerical control component is determined based on the component feature.

The replacement necessity degree refers to a necessity degree to which the gas numerical control component needs to be replaced. In some embodiments, the replacement necessity degree may be represented by a numerical value from 0 to 1, where a higher value indicates a higher replacement necessity degree.

In some embodiments, the processor may determine the replacement necessity degree based on the component feature in various ways.

For example, the processor may construct a current first vector based on a current data processing feature, a current working feature, and a current environmental feature; retrieve a historical first vector with the highest first similarity to the current first vector from a first vector database, use it as a reference first vector, the reference first vector being composed of a reference data processing feature, a reference working feature, and a reference environmental feature; in response to the first similarity between the current first vector and the reference first vector being greater than a first similarity threshold, if any component in the current first vector is smaller than the corresponding component in the reference first vector, use the first similarity as a current replacement necessity degree; if there is a component in the current first vector greater than the corresponding component in the reference first vector, replace that one or more components in the current first vector with the corresponding component(s) of the reference first vector to reconstruct the current first vector; and repeat the above steps until the current replacement necessity degree is determined.

The comparison of the magnitude of components in the current first vector with corresponding components in the reference first vector includes comparing the magnitude of the current data processing feature with the reference data processing feature, comparing the magnitude of the current working feature with the reference working feature, and comparing a severity degree of the current environmental feature with a severity degree of the reference environmental feature. The severity degree may be represented by a difference between an actual environmental feature and an ideal environmental feature, where a larger absolute value of the difference indicates a higher severity degree. For example, the severity degree of the reference environmental feature may be represented by a difference between a historical actual environmental feature and a historical ideal environmental feature.

In some embodiments, the first vector database may be constructed by the processor or technical personnel based on historical data, and the first vector database includes historical first vectors of a plurality of replaced historical gas numerical control components and corresponding historical replacement necessity degrees.

In some embodiments, the first similarity may be represented by Euclidean distance, cosine similarity, etc. The first similarity threshold may be set by default by the processor or preset by the technical.

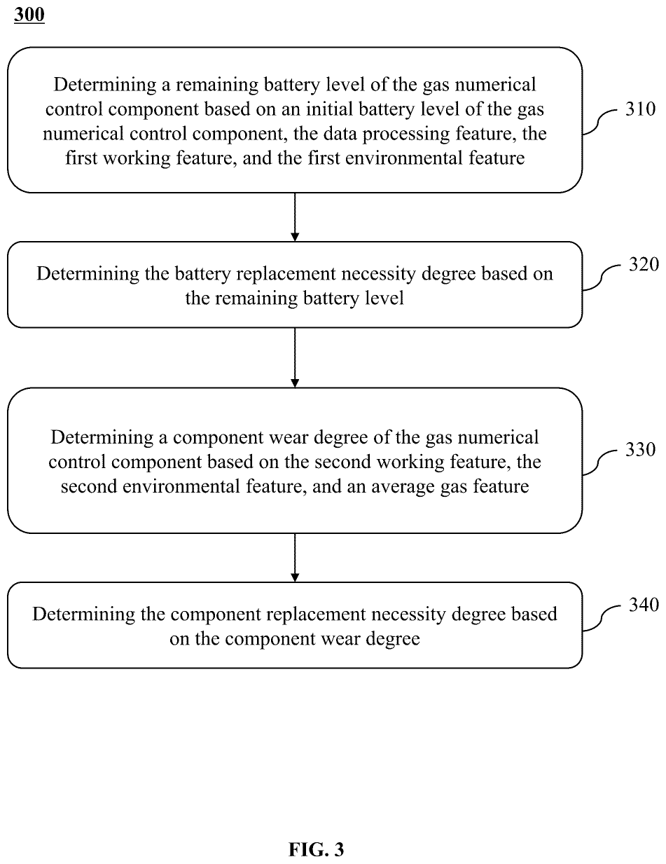

In some embodiments, the processor may also determine a remaining battery level of the gas numerical control component based on an initial battery level, the data processing feature, a first working feature, and a first environmental feature of the gas numerical control component, thereby determining a battery replacement necessity degree; and determine a component wear degree based on a second working feature, a second environmental feature, and an average gas feature, thereby determining a component replacement necessity degree. More details about this part may be found in FIG. 3 and related descriptions.

In 230, a replacement parameter is determined based on the replacement necessity degree.

The replacement parameter refers to a relevant parameter regarding the replacement situation of the gas numerical control component. In some embodiments, the replacement parameter may include a component to be replaced and a replacement manner of the component to be replaced.

The component to be replaced refers to a gas numerical control component determined to need replacement.

The replacement manner refers to a manner of replacing the gas numerical control component. In some embodiments, the replacement manner may include battery replacement and overall component replacement.

In some embodiments, the replacement parameter includes replacement sub-parameters of a plurality of gas regions within the gas pipeline network. The processor may determine the replacement parameter (e.g., the replacement sub-parameters) based on the replacement necessity degree in various ways. For example, the first vector database may further include the historical first vectors of the plurality of replaced historical gas numerical control components, the corresponding historical replacement necessity degrees, and historical replacement manners; the processor may take a gas numerical control component whose replacement necessity degree is higher than a reference replacement necessity degree as the component to be replaced; retrieve and determine the reference first vector of the component to be replaced in the first vector database, and take the historical replacement manner corresponding to the reference first vector as the replacement manner of the component to be replaced.

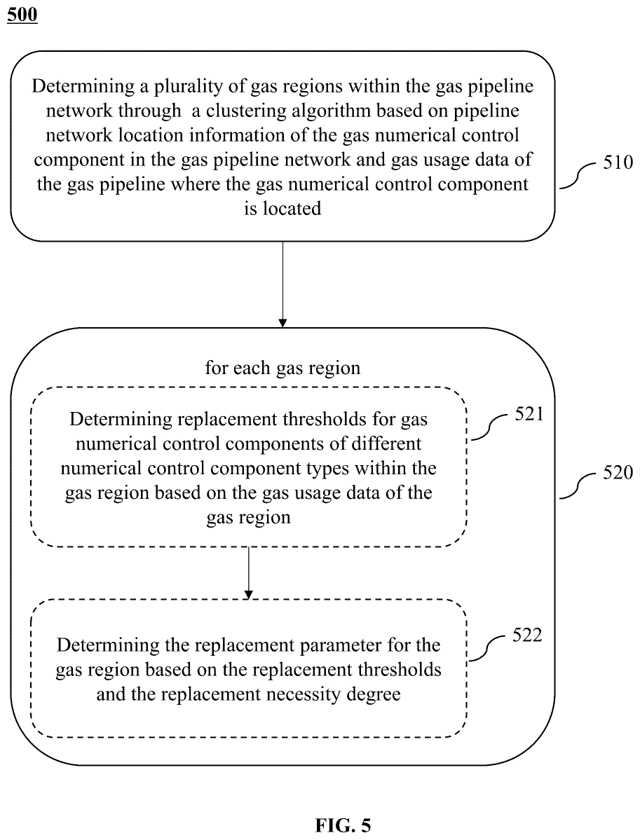

In some embodiments, the processor may statistically determine a minimum value of the historical replacement necessity degrees of a plurality of historical gas numerical control components in the first vector database and use it as the reference replacement necessity degree. In some embodiments, the processor may also determine replacement parameters (i.e., replacement sub-parameters) of a plurality of gas regions within the gas pipeline network based on pipeline network location information and the replacement necessity degree of the gas numerical control component in the gas pipeline network. More details about this part may be found in FIG. 5 and related descriptions.

In 240, a replacement task instruction and a gas shutoff parameter are generated based on the replacement parameter, the replacement task instruction is transmitted to the maintenance user sub-platform via the maintenance service sub-platform, and the gas shutoff parameter is transmitted to the government safety supervision management platform via the government safety supervision sensing network platform.

The replacement task instruction refers to an instruction to perform a replacement task on the component to be replaced. In some embodiments, the replacement task instruction may include the component to be replaced, the replacement manner of the component to be replaced, and a replacement time period of the component to be replaced.

In some embodiments, the replacement parameter may also include the replacement time period for the component to be replaced. The replacement time period may be determined by the processor based on a queuing situation of pending replacement tasks. For example, if a queuing duration for the pending replacement tasks is 8 hours, the replacement time period for the replacement task is a certain period after 8 hours. A duration of the replacement time period is represented by a product of a total count of components to be replaced in the replacement task and an average replacement duration per component. The average replacement duration per single component may be statistically obtained by the processor or technical personnel based on historical data.

In some embodiments, the processor may autonomously generate the replacement task instruction based on the replacement parameter according to a preset template.

The gas shutoff parameter refers to a relevant parameter regarding gas supply shutoff. In some embodiments, the gas shutoff parameter may include a gas shutoff pipeline and a gas shutoff time period.

The gas shutoff pipeline refers to a gas pipeline where the gas supply is stopped. The gas shutoff time period refers to a time period during which the gas supply is stopped.

In some embodiments, the processor may generate the gas shutoff parameter based on the replacement parameter according to a preset rule. For example, the processor may take a gas pipeline where the component to be replaced is located as the gas shutoff pipeline, and take the replacement time period of the component to be replaced as the gas shutoff time period.

In some embodiments, the processor may transmit the replacement task instruction to the maintenance user sub-platform via the maintenance service sub-platform, so that maintenance personnel perform the replacement task on the component to be replaced in response to the replacement task instruction. The processor may also transmit the gas shutoff parameter to the government safety supervision management platform via the government safety supervision sensing network platform, and the government safety supervision management platform determines and generates a shutoff parameter confirmation instruction.

In 250, in response to receiving the shutoff parameter confirmation instruction from the government safety supervision management platform, a gas shutoff instruction is generated, and the gas shutoff instruction is transmitted to a gas supply control device via the gas company sensing network platform to control the open/closed state of the gas supply control device.

The shutoff parameter confirmation instruction refers to an instruction confirming the gas shutoff parameter. In some embodiments, the shutoff parameter confirmation instruction may include a confirmed gas shutoff pipeline and a confirmed gas shutoff time period.

In some embodiments, after receiving the replacement task instruction, the maintenance personnel may adjust the replacement time period in the replacement parameter on the maintenance user sub-platform according to the actual situation (e.g., if a pending replacement task is canceled from the queue, the replacement time period may be appropriately advanced); the maintenance user sub-platform sends an adjusted replacement parameter to the government safety supervision management platform, and the government safety supervision management platform automatically updates the gas shutoff time period in the gas shutoff parameter based on the adjusted replacement parameter to determine the shutoff parameter confirmation instruction.

The gas shutoff instruction refers to an instruction to stop the gas supply in the gas pipeline. In some embodiments, the gas shutoff instruction may include the gas supply control device corresponding to the gas shutoff pipeline and a closing time period of the gas supply control device.

In some embodiments, the processor may obtain the shutoff parameter confirmation instruction via the government safety supervision management platform, determine the gas supply control device corresponding to the gas shutoff pipeline based on the gas shutoff pipeline in the shutoff parameter confirmation instruction, determine the closing time period of the gas supply control device based on the gas shutoff time period, thereby determining the gas shutoff instruction; and transmit the gas shutoff instruction to the gas supply control device via the gas company sensing network platform to control the open/closed state of the gas supply control device.

The gas supply control device being in an open state represents that the gas pipeline is supplying gas, and the gas supply control device being in a closed state represents that the gas pipeline has stopped supplying gas.

More content regarding the gas supply control device may be found in the related description of FIG. 1.

In some embodiments, the processor may control the open/closed state of the gas supply control device based on the gas shutoff time period in the gas shutoff instruction. For example, if the gas shutoff pipeline is a gas pipeline L and its gas shutoff time period is October 20th, 13:00-15:00, then the gas supply control device of the gas pipeline L is closed at 13:00 on October 20th and opened at 15:00.

In 260, gas shutoff reminder information is generated, and the gas shutoff reminder information is transmitted to the gas user sub-platform and the citizen user platform.

In some embodiments, the form of the gas shutoff reminder information may include phone notification, SMS notification, TV notification, gas shutoff reminder sign, etc.

In some embodiments, the processor may autonomously generate the gas shutoff reminder information based on the shutoff parameter confirmation instruction.

In some embodiments, the processor may transmit the gas shutoff reminder information to the gas user sub-platform in advance based on the gas shutoff time period in the shutoff parameter confirmation instruction to remind gas users to prepare for the gas shutoff in advance. The processor may also transmit the gas shutoff reminder information to the citizen user platform to increase notification channels, further expand notification coverage, and improve the probability of the gas users receiving the gas shutoff reminder information.

In 270, in response to receiving a replacement completion message issued by the maintenance user sub-platform, a replaced component is determined, and the data partition corresponding to the replaced component is updated.

The replacement completion message refers to notification information that the component to be replaced has been replaced.

In some embodiments, after completing the replacement of the component to be replaced, the maintenance personnel may perform a replacement completion operation on the maintenance user sub-platform (e.g., click a battery replacement completion button, input the replaced component, etc.), and the maintenance user sub-platform may generate the replacement completion message and send it to the gas company management platform via the maintenance service sub-platform.

The replaced component refers to a gas numerical control component that has replaced the component to be replaced.

In some embodiments, the processor may determine the replaced component and update the data partition corresponding to the replaced component based on the replacement completion message issued by the maintenance user sub-platform.

It should be noted that if the replacement manner of the completed replacement of the component to be replaced is battery replacement, the data partition corresponding to the component to be replaced is compressed and stored to save data partition space; if the replacement manner of the completed replacement of the component to be replaced is overall component replacement, the data partition is cleared and reset to avoid unlimited data accumulation and increased system operation load.

According to some embodiments of the present disclosure, by determining the necessity degree for replacing the gas numerical control component based on the component feature, and then determining the component to be replaced and the corresponding replacement manner to generate the replacement task instruction and the gas shutoff instruction for executing component replacement, gas supply shutoff, and gas shutoff reminder, etc., dynamic monitoring and intelligent decision-making for the replacement of the gas pipeline network component can be achieved, thereby improving the safety and efficiency of gas supply, and ensuring the stable operation of the gas pipeline network.

FIG. 3 is a flowchart illustrating an exemplary process 300 for determining a replacement necessity degree according to some embodiments of the present disclosure. In some embodiments, the process 300 may be executed by the gas company management platform 141. For example, the process 300 may be executed by a processor in the gas company management platform 141.

In some embodiments, a working feature may include a first working feature and a second working feature; the first working feature is the working feature of a gas numerical control component during a first time period, the second working feature is the working feature of the gas numerical control component during a second time period; an environmental feature may include a first environmental feature and a second environmental feature; the first environmental feature is the environmental feature of the gas numerical control component during the first time period, the second environmental feature is the environmental feature of the gas numerical control component during the second time period; the first time period is a time period from a historical time point of the last battery replacement to a current time point, the second time period is a time period from a historical time point of the last component replacement to the current time point.

The historical time point of the last battery replacement and the historical time point of the last component replacement may be determined based on an upload time of the historical replacement completion message.

In some embodiments, the replacement necessity degree may include a battery replacement necessity degree and a component replacement necessity degree.

The battery replacement necessity degree refers to a necessary degree of performing battery replacement on the gas numerical control component. The component replacement necessity degree refers to a necessary degree of performing overall replacement on the gas numerical control component.

In some embodiments, both the battery replacement necessity degree and the component replacement necessity degree may be represented by a numerical value from 0 to 1. The higher the value, the higher the battery replacement necessity degree and the component replacement necessity degree.

As shown in FIG. 3, the process 300 includes the following operations 310-340.

In 310, a remaining battery level of the gas numerical control component is determined based on an initial battery level, the data processing feature, the first working feature, and the first environmental feature of the gas numerical control component.

In some embodiments, the processor may search in a power consumption database to determine a historical first environmental feature with the highest similarity to a current first environmental feature, take it as a reference first environmental feature, and determine a historical power consumption speed corresponding to the reference first environmental feature as a power consumption speed of the current gas numerical control component; determine a product of a data collection frequency and a data collection amount in the data processing feature of the current gas numerical control component, and a start-stop frequency and a working duration in the first working feature as a data collection total amount of the gas numerical control component; determine a product of a data transmission frequency and a data transmission amount in the data processing feature of the current gas numerical control component, and the start-stop frequency and the working duration in the first working feature as a data transmission total amount of the gas numerical control component; determine a sum of the data collection total amount and the data transmission total amount multiplied by the power consumption speed as a battery power consumption amount; and determine a difference obtained by subtracting the battery power consumption amount from the initial battery level as the remaining battery level.

The power consumption database includes historical first environmental features and corresponding historical power consumption speeds of the plurality of replaced historical gas numerical control components.

The power consumption speed refers to a battery power consumed to collect and/or transmit unit data. The historical power consumption speed in the power consumption database may be represented by a ratio of a historical battery power consumption amount of a historical gas numerical control component to a historical data total amount under the historical first environmental feature. The historical data total amount is a sum of a historical data collection total amount and a historical data transmission total amount.

In 320, the battery replacement necessity degree is determined based on the remaining battery level.

In some embodiments, the processor may construct a current second vector based on the remaining battery level, the working feature, and the environmental feature, search in a second vector database to determine a historical second vector with the highest second similarity to the current second vector, take it as a reference second vector, and determine a historical remaining working duration corresponding to the reference second vector as a remaining working duration of the battery of the current gas numerical control component. The battery replacement necessity degree is negatively correlated with the remaining working duration, and the shorter the remaining working duration, the higher the battery replacement necessity degree.

In some embodiments, the second vector database may be constructed by the processor or a technician based on historical data, and the second vector database includes historical second vectors and corresponding historical remaining working durations of the plurality of historical gas numerical control components. Regarding the setting of the second similarity, reference may be made to the first similarity.

In 330, a component wear degree of the gas numerical control component is determined based on the second working feature, the second environmental feature, and an average gas feature.

The average gas feature refers to an average amount of gas-related parameters. For example, the average gas feature may include an average gas amount, an average gas flow rate, an average gas temperature, an average gas pressure, and an average gas impurity content. In some embodiments, the gas numerical control component may collect gas-related parameters, and the processor calculates the average gas feature based on the gas-related parameters.

The component wear degree refers to a degree of performance degradation or function loss of the component. In some embodiments, the component wear degree may be represented by a percentage between 0 and 1. The larger the percentage, the higher the component wear degree.

In some embodiments, the processor may determine the component wear degree of the gas numerical control component in various ways based on the second working feature, the second environmental feature, and the average gas feature.

For example, the processor may construct a current third vector based on the second working feature, the second environmental feature, and the average gas feature, search in a third vector database for a historical third vector with the highest third similarity to the current third vector, take it as a reference third vector, the reference third vector being constituted by a reference second working feature, a reference second environmental feature, and a reference average gas feature; and determine a historical component wear degree corresponding to the reference third vector as the component wear degree of the current gas numerical control component.

In some embodiments, the third vector database includes historical third vectors and corresponding historical component wear degrees of the plurality of replaced historical gas numerical control components. The historical component wear degree may be obtained by the processor through deep calculation based on historical actual wear data uploaded by the maintenance personnel. For example, the processor may determine the historical component wear degree based on historical actual wear pictures of historical gas numerical control components using a trained machine learning model. Regarding the setting of the third similarity, reference may be made to the first similarity.

In some embodiments, the processor may also determine a wear coefficient of the gas numerical control component based on the second working feature, the second environmental feature, the average gas feature, and reference usage data; and determine the component wear degree based on a component usage duration, a design service life, and the wear coefficient of the gas numerical control component.

The reference usage data refers to relevant data of the gas numerical control components whose service life has reached the design service life, including the reference second working feature, the reference second environmental feature, and the reference average gas feature. In some embodiments, the reference usage data may be obtained based on historical data.

The wear coefficient refers to a coefficient that measures the wear speed of the gas numerical control component. In some embodiments, the faster the gas numerical control component wears, the higher the wear coefficient.

In some embodiments, the processor may respectively calculate ratios of the second working feature, the second environmental feature, and the average gas feature of the gas numerical control component to the second working feature, the second environmental feature, and the average gas feature in the reference usage data, to obtain a working feature ratio, an environmental feature ratio, and a gas feature ratio; and perform a weighted summation on the three ratios according to load weights to obtain the wear coefficient.

The load weight may characterize the degree of influence of different feature data on the wear coefficient. For example, if the environment has the greatest influence on the wear coefficient, the environmental feature ratio has the largest load weight. In some embodiments, the load weights for different feature ratios may be preset by the system or a technician.

In some embodiments, the processor may also determine the load weights through a control variable manner. The processor may group the plurality of replaced historical gas numerical control components by controlling variables. The variables may include the second working feature, the second environmental feature, and the average gas feature. The processor may group historical gas numerical control components that share two identical variables and one different variable into one group, that is, the plurality of historical gas numerical control components may be divided into 3 groups, each group of historical gas numerical control components corresponding to one different variable. The processor may calculate a variable influence value of the different variables corresponding to each group of historical gas numerical control components and use a ratio of the variable influence values as a ratio of the load weights, with a sum of the load weights being 1.

For example, the processor may group the historical gas numerical control components with the same second working feature and second environmental feature but different average gas features into one group, where the different variable corresponding to the group of historical gas numerical control components is the average gas feature; determine a first difference between a maximum average gas feature and a minimum average gas feature among the average gas features (e.g., average gas temperatures) of the group of historical gas numerical control components, and determine a second difference between an actual service life of the historical gas numerical control component corresponding to the maximum average gas feature and an actual service life of the historical gas numerical control component corresponding to the minimum average gas feature; determine a ratio of the first difference to the second difference as a variable influence value of the average gas feature; similarly determine variable influence values of the second working feature and the second environmental feature; determine ratios of the variable influence values of the second working feature, the second environmental feature, and the average gas feature as ratios of the load weights of the working feature ratio, the environmental feature ratio, and the gas feature ratio; and determine the load weights of the working feature ratio, the environmental feature ratio, and the gas feature ratio in combination with the sum of the load weights being 1.

The component usage duration refers to an actual operating time of the gas numerical control component from the start of use to the current time point.

The design service life refers to a preset normal working duration of the gas numerical control component in factory settings.

In some embodiments, the processor may determine the component wear degree based on the component usage duration, the design service life, and the wear coefficient of the gas numerical control component. Exemplarily, the component wear degree may be calculated using the following formula (1):

$$W = C * \frac{T}{U} , \qquad ( 1 )$$

where W denotes the component wear degree, C denotes the wear coefficient, T denotes the component usage duration, and U denotes the design service life.

In some embodiments of the present disclosure, by leveraging the second working feature, the second environmental feature, the average gas feature, and the reference usage data, and comprehensively accounting for the importance and influence of each factor, the wear coefficient can be calculated more precisely, and then combined with the component usage duration and the design service life, the wear degree of the gas numerical control component can be accurately evaluated.

In 340, the component replacement necessity degree is determined based on the component wear degree.

In some embodiments, the processor may determine the component replacement necessity degree based on the component wear degree in various ways. For example, the processor may calculate a difference between the component wear degree and an average historical wear degree. The larger the difference, the higher the component replacement necessity degree.

The average historical wear degree refers to an average value of historical component wear degrees in the historical replacement record.

In some embodiments of the present disclosure, by comprehensively analyzing the working feature, the environmental feature, the data processing feature, and the average gas feature of the gas numerical control component, the remaining battery level and the component wear degree can be accurately evaluated, thereby determining the battery replacement necessity degree and the component replacement necessity degree, providing data support for making reasonable replacement decisions subsequently, which is beneficial for optimizing maintenance costs and ensuring the safe operation of the gas pipeline network.

In some embodiments, the processor may also acquire gas monitoring data collected by the gas numerical control component from the data center; determine a collection confidence level of the gas numerical control component based on the gas monitoring data; determine an operational stability level of the gas numerical control component based on a plurality of collection confidence levels corresponding to the gas numerical control component at a plurality of time points; and adjust the component replacement necessity degree based on the collection confidence level and the operational stability level.

More descriptions regarding the gas numerical control component, the data center, the gas monitoring data, and the component replacement necessity degree may be found in FIGS. 2-3.

The gas monitoring data refers to data collected by the gas numerical control component, e.g., gas flow rate, gas pressure, gas temperature, etc.

In some embodiments, the gas numerical control component may upload the collected gas monitoring data to the data center of the gas company management platform via the gas company sensing network platform, and the processor may directly retrieve the gas monitoring data from the data center.

The collection confidence level refers to a credibility degree of the gas numerical control component in performing collection work. In some embodiments, the collection confidence level may be represented by a numerical value from 0 to 1. The larger the value, the higher the collection confidence level of the gas numerical control component, and the more reliable the gas monitoring data collected by the gas numerical control component.

In some embodiments, the processor may determine data differences between the same gas monitoring data collected at the same time point by the plurality of gas numerical control components located on the same reference gas pipeline; perform a plurality of verifications on the plurality of gas numerical control components based on the data differences and a reference difference range to determine a verification result; and determine the collection confidence level based on the verification result.

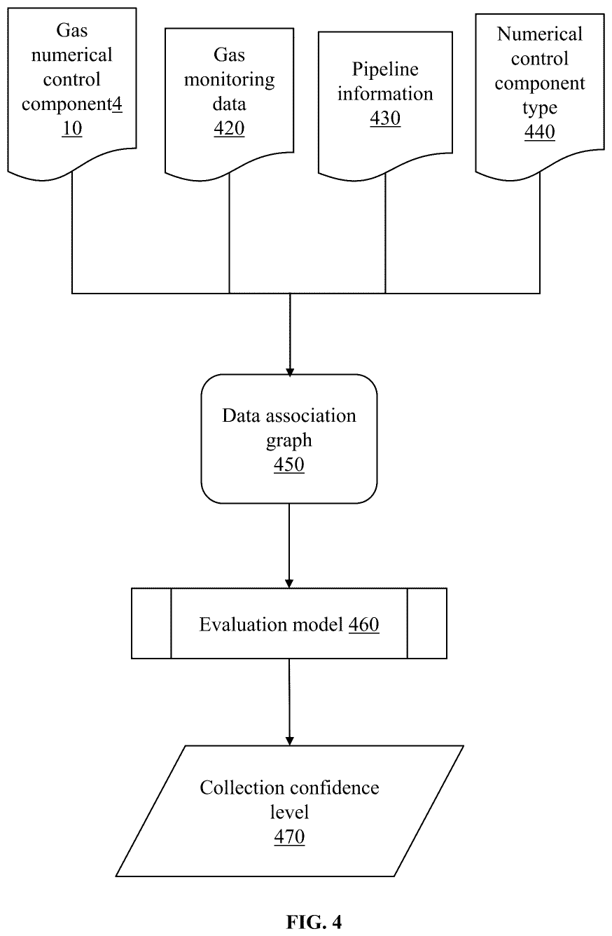

The reference difference range refers to a difference range between gas monitoring data monitored by upstream and downstream gas numerical control components on the same reference gas pipeline under normal conditions of the gas numerical control components. In some embodiments, the reference difference range may vary depending on a numerical control component type and an interval pipeline length. In some embodiments, the processor may determine the reference difference range based on historical data. More descriptions regarding the numerical control component type and the interval pipeline length may be found in FIG. 4 and the related descriptions.

In some embodiments, the data difference may refer to a difference obtained by subtracting gas monitoring data of a downstream gas numerical control component from gas monitoring data of an upstream gas numerical control component among two adjacent gas numerical control components.

Merely by way of example, gas numerical control components A₁, A₂, A₃, A₄, and A₅ are gas flow sensors located on the same reference gas pipeline S₁ and deployed sequentially from upstream to downstream. The gas flow rates collected at the same time point are f₁, f₂, f₃, f₄, and f₅ respectively, the corresponding data differences are f₁₋₂, f₂₋₃, f₃₋₄, and f₄₋₅ respectively, and the corresponding reference difference ranges are (0,j₁₋₂), (0,j₂₋₃), (0,j₃₋₄), and (0,j₄₋₅) respectively. If f₂₋₃ is not within (0,j₂₋₃) and f₃₋₄ is not within (0,j₃₋₄), it is verified that gas numerical control components A₂, A₃, A₄ may have abnormalities; while f₁₋₂ is within (0,j₁₋₂), verifying that gas numerical control component A₂ has no abnormality, and f₄₋₅ is within (0,j₄₋₅), verifying that gas numerical control component A₄ has no abnormality, then the verification results of the plurality of verifications comprehensively indicates that gas numerical control component A₃ may be abnormal, the collection confidence level of gas numerical control component A₃ is lower, and the collected gas monitoring data f₃ is less reliable.

FIG. 4 is an exemplary schematic diagram of an evaluation model according to some embodiments of the present disclosure.

In some embodiments, as shown in FIG. 4, the processor may construct a data association graph 450 based on a plurality of gas numerical control components 410 located on a same reference gas pipeline, gas monitoring data 420 collected by the plurality of gas numerical control components, pipeline information 430 between the plurality of gas numerical control components, and numerical control component types 440 of the plurality of gas numerical control components; and determine a collection confidence level 470 based on the data association graph 450 using an evaluation model 460.

The pipeline information between the gas numerical control components refers to related information about the gas pipeline between the gas numerical control components, e.g., an interval pipeline length, a pipeline inner diameter, a pipeline cleaning time, etc.

The interval pipeline length refers to a length of the gas pipeline between two gas numerical control components. In some embodiments, the interval pipeline length may be determined by the processor based on installation location coordinates of the gas numerical control components and a gas pipeline network distribution map. For example, the processor may mark the gas numerical control components in the gas pipeline network distribution map according to the installation location coordinates, and measure the length of the gas pipeline between two gas numerical control components, which is the interval pipeline length. The installation location coordinates of the gas numerical control components may be acquired by a positioning device of the gas numerical control components, or may be acquired from installation record data during installation.

The pipeline inner diameter refers to a diameter of an internal cross-section of the gas pipeline. The pipeline cleaning time refers to a time it takes to clean the pipeline. In some embodiments, the gas pipeline network distribution map, the pipeline inner diameter, and the pipeline cleaning time may all be acquired by the processor from the data center.

The numerical control component type refers to the type of the gas numerical control component. For example, the gas numerical control component may be classified into various numerical control component types according to function, such as a temperature numerical control component, a flow data component, etc.

The data association graph refers to a graph showing relationships among the gas numerical control components, the gas monitoring data, the pipeline information, and the numerical control component types. In some embodiments, the data association graph is composed of a plurality of nodes and a plurality of edges.

The plurality of nodes of the data association graph are the plurality of gas numerical control components located on the same reference gas pipeline, one node corresponds to one gas numerical control component, and a node feature is the gas monitoring data and the numerical control component type of the gas numerical control component.

The edge of the data association graph is the gas pipeline between the gas numerical control components, and an edge feature is the pipeline information between the gas numerical control components.

In some embodiments, the plurality of gas numerical control components on one gas pipeline and the gas pipelines between the plurality of gas numerical control components constitute the data association graph.

The evaluation model refers to a model used to determine the collection confidence level. In some embodiments, the evaluation model may be a machine learning model, such as a Graph Neural Network (GNN), etc.

In some embodiments, as shown in FIG. 4, an input to the evaluation model 460 may be the data association graph 450, and an output may be the collection confidence level 470 of the nodes of the data association graph. The data association graph 450 may be determined by the processor based on the gas numerical control components 410, the gas monitoring data 420, the pipeline information 430, and the numerical control component types 440.

In some embodiments, the evaluation model may be trained and acquired in various ways. For example, the evaluation model may be trained and acquired using a plurality of training samples with training labels. The training sample for training the evaluation model may be a sample data association graph, and a training label corresponding to the training sample is an actual collection confidence level of sample gas numerical control components corresponding to a plurality of sample nodes in the sample data association graph.

In some embodiments, the processor may acquire the plurality of training samples based on historical data. One historical data association graph may serve as one training sample. For each sample gas numerical control component in each training sample, the processor may acquire, from the data center, a historical gas numerical control component that is of the same type as the sample gas numerical control component and has accurate data, use it as a reference gas numerical control component for the sample gas numerical control component, and acquire a plurality of reference gas monitoring data collected by the reference gas numerical control component at the same location and the same time; calculate a plurality of differences between the plurality of sample gas monitoring data of the sample gas numerical control component and the plurality of reference gas monitoring data of the reference gas numerical control component; if the difference is within an allowable error range, the sample gas monitoring data is considered accurate; the processor determines a ratio of a count of accurate sample gas monitoring data to a count of all sample gas monitoring data as the actual collection confidence level. The allowable error range may be acquired from factory setting values of the gas numerical control component.

For example, the plurality of sample gas monitoring data of the sample gas numerical control component include sample gas temperature, sample gas flow, sample gas pressure, and the plurality of reference gas monitoring data of the reference gas numerical control component include reference gas temperature, reference gas flow, and reference gas pressure. If a difference between the sample gas temperature and the reference gas temperature is within the allowable error range, the sample gas temperature is accurate; and the gas flow and the sample gas pressure determined similarly are inaccurate, the actual collection confidence level of the sample gas numerical control component is ⅓.

In some embodiments, after replacing a component to be replaced, the processor may determine a collection confidence level of a replaced component using the evaluation model; in response to that, after the replacement is performed, there are still more than a preset count of gas numerical control components whose collection confidence levels are all less than a preset confidence level threshold, it is determined that an output result of the evaluation model is not accurate enough, and therefore an incremental training set needs to be generated to incrementally update the evaluation model.

The processor may collect the gas monitoring data a plurality of times based on the gas numerical control components; reconstruct a large count of data association graphs as incremental training samples; adjust the collection confidence level of the replaced component to a reference confidence level; keep collection confidence levels of other unreplaced gas numerical control components unchanged to obtain labels corresponding to the incremental training samples; and perform incremental training on a current evaluation model based on the incremental training set composed of a large count of incremental training samples and their labels. The training manner may refer to the training process described above. The preset count and the preset confidence level threshold may both be preset by a technician. The reference confidence level refers to an average value of the collection confidence levels of the gas numerical control components with accurate gas monitoring data.

In some embodiments of the present disclosure, by using the plurality of gas numerical control components on the same reference gas pipeline, combining various data and information to construct the data association graph, and based on the data association graph, accurately and efficiently determining the collection confidence levels of the plurality of gas numerical control components through the machine learning model, the reliability of data collected by the gas numerical control components can be effectively evaluated, so as to reasonably determine the component replacement necessity degree subsequently.

The operational stability level refers to a stable degree of operation of the gas numerical control component.

In some embodiments, the processor may determine the operational stability level of the gas numerical control component based on the plurality of collection confidence levels corresponding to the gas numerical control component at a plurality of time points through various ways. For example, the processor may determine a stable duration of the gas numerical control component based on a plurality of confidence fluctuation values of the collection confidence levels of the gas numerical control component at the plurality of time points relative to reference confidence levels at the plurality of time points; and determine the operational stability level based on the stable duration and the plurality of confidence fluctuation values at the plurality of time points.

The confidence fluctuation value refers to a range of fluctuation of the collection confidence level relative to the reference confidence level. For example, the confidence fluctuation value may be a difference between the collection confidence level and the reference confidence level.

The stable duration refers to a length of time during which the collection confidence level remains stable. In some embodiments, the processor may determine the plurality of confidence fluctuation values corresponding to the collection confidence levels at the plurality of time points; take a longest time period constituted by the plurality of consecutive time points where the confidence fluctuation values are continuously positive as a stable period, and take a mean duration of a plurality of stable periods as the stable duration.

In some embodiments, the longer the stable duration and the smaller the average value of the confidence fluctuation values, the higher the operational stability level. For example, the processor may calculate the operational stability level based on the following formula (2):

$$R = k \times t \div α , \qquad ( 2 )$$

where R denotes the operational stability level, k denotes a stability coefficient, t denotes the stable duration, a denotes a mean value of the plurality of confidence fluctuation values, and the stability coefficient k may be preset by the processor or a technician.

In some embodiments, the processor adjusts the component replacement necessity degree based on the collection confidence level and the operational stability level through various ways. For example, in response to the collection confidence level being lower than the preset confidence level threshold, the processor may increase the component replacement necessity degree, and an increase amplitude of the component replacement necessity degree may be consistent with a difference amplitude between the collection confidence level and the preset confidence level threshold. As another example, in response to the collection confidence level being higher than the confidence level threshold and the operational stability level being lower than a preset stability threshold, the processor may increase the component replacement necessity degree, and the increase amplitude of the component replacement necessity degree may be consistent with a difference amplitude between the operational stability level and the preset stability threshold. The preset stability threshold may be preset by the processor or a technician.

In some embodiments of the present disclosure, by accurately evaluating the collection confidence level of the gas numerical control component through the gas monitoring data, then determining the operational stability level of the gas numerical control component based on the collection confidence levels at the plurality of time points, and further comparing the collection confidence level and the operational stability level with corresponding preset values to reasonably adjust the component replacement necessity degree, the component replacement necessity degree becomes more reliable and accurate, which facilitates accurately determining whether the gas numerical control component needs to be replaced, and saving replacement costs while ensuring normal operation of the gas numerical control component.

In some embodiments, the processor may determine replacement parameters (i.e., replacement sub-parameters) of a plurality of gas regions within the gas pipeline network based on pipeline network location information and the replacement necessity degree of the gas numerical control component in the gas pipeline network.

More descriptions regarding the gas numerical control component, the replacement necessity degree, and the replacement parameter may be found in FIGS. 2-4.

The pipeline network location information refers to the location information of the gas numerical control component in the gas pipeline network. In some embodiments, the pipeline network location information of the gas numerical control component may include a pipeline level where the gas numerical control component is located and the installation location coordinates.

The pipeline level where the gas numerical control component is located refers to a division level of the gas pipeline where the gas numerical control component is located. For example, the pipeline level may include a main pipeline, a primary branch, a secondary branch, etc. In some embodiments, the pipeline level where the gas numerical control component is located may be directly acquired by the processor from the data center.

More descriptions regarding the installation location coordinates may be found in FIG. 4.

The gas region refers to a sub-region in the gas pipeline network. In some embodiments, the gas pipeline network may be divided into a plurality of gas regions, and the division of the gas regions may be performed by a technician or by the processor according to a preset rule (e.g., an equal-area rule).

In some embodiments, the processor may determine the replacement parameters of the plurality of gas regions within the gas pipeline network based on the pipeline network location information and the replacement necessity degree of the gas numerical control component in the gas pipeline network in various ways. For example, the processor may respectively determine a reference necessity degree for the location information based on historical replacement records of gas numerical control components corresponding to different location information; and determine whether the gas numerical control component needs to be replaced and a replacement manner according to the reference necessity degree corresponding to the location information and the replacement necessity degree of the gas numerical control component. The determination manner may be consistent with the determination manner in operation 220 of FIG. 2. More descriptions regard this part may be found in FIG. 2 and related descriptions.

FIG. 5 is a flowchart illustrating an exemplary process 500 for determining a replacement parameter of a gas region according to some embodiments of the present disclosure. In some embodiments, the process 500 may be implemented by the IoT system 100, and may be executed by the gas company management platform 141 within the IoT system 100. For example, the process 500 may be executed by a processor within the gas company management platform 141. As shown in FIG. 5, the process 500 includes the following operations 510-520.

In 510, a plurality of gas regions of the gas pipeline network are determined through a clustering algorithm based on pipeline network location information of the gas numerical control component in the gas pipeline network and gas usage data of the gas pipeline where the gas numerical control component is located.

The gas usage data refers to data related to gas usage by gas users. For example, the gas usage data of the gas pipeline where the gas numerical control component is located may include a total gas usage amount of the gas users on the gas pipeline where the gas numerical control component is located, a gas usage duration of the gas users on the gas pipeline where the gas numerical control component is located, etc. In some embodiments, the processor may acquire the gas usage data based on the gas user platform.

In some embodiments, the clustering algorithm may be a clustering algorithm with a given count of clusters, such as K-means clustering algorithm and its derivative clustering algorithms, etc.

In some embodiments, the processor determines the plurality of gas regions of the gas pipeline network through the clustering algorithm based on the pipeline network location information of the gas numerical control component in the gas pipeline network and the gas usage data of the gas pipeline where the gas numerical control component is located. Taking the K-means clustering algorithm as an example, the processor may constitute a fourth vector based on the pipeline network location information of the gas numerical control component and the gas usage data of a pipeline where the gas numerical control component is located. The plurality of gas numerical control components correspond to a plurality of fourth vectors. The processor may randomly determine K cluster centers from the plurality of fourth vectors; for each fourth vector, calculate its vector distance to the K cluster centers respectively, and assign it to the cluster of the cluster center with the closest vector distance; traverse all fourth vectors to form K clusters; for each cluster, calculate a mean value of the fourth vectors within the cluster, and use the mean value as a new cluster center for the cluster; repeat the above steps until the cluster centers no longer change. After the clustering is completed, K clusters are determined, and the pipeline network location information of the gas components within each cluster corresponds to and constitutes the respective gas regions.

The count of cluster centers, i.e., the aforementioned K value, may be set by the processor by default or by a technician based on experience. For example, a large count of historical replacement thresholds of historical gas numerical control components in historical data are clustered using a clustering algorithm without pre-specifying the count of clusters, and the count of clusters formed by clustering is determined as the K value. The clustering algorithm without pre-specifying the count of clusters may include a density-based spatial clustering of applications with noise (DBSCAN) clustering algorithm, etc.

In some embodiments, for each gas region, the operation 520 may include operations 521-522.

In 521, replacement thresholds for gas numerical control components of different numerical control component types within the gas region are determined based on the gas usage data of the gas region.

The replacement threshold refers to a minimum value of the replacement necessity degree of a gas numerical control component that needs to be replaced. When the replacement necessity degree of the gas numerical control component is greater than or equal to the replacement threshold, the gas numerical control component is a component to be replaced. In some embodiments, the replacement threshold may include a battery replacement threshold and a component replacement threshold.

The battery replacement threshold refers to a minimum value of the battery replacement necessity degree for the component to be replaced. The component replacement threshold refers to a minimum value of the component replacement necessity degree for the component to be replaced.

In some embodiments, the replacement thresholds for gas numerical control components of the same numerical control component type may be different in different gas regions, and the replacement thresholds for gas numerical control components of different numerical control component types within the same gas region may also be different.

In some embodiments, for each gas region, the processor may determine the replacement thresholds for gas numerical control components of different numerical control component types within that gas region based on the gas usage data of the gas region in various ways. For example, for a certain gas region, the processor may calculate a mean value of historical battery replacement necessity degrees and a mean value of historical component replacement necessity degrees of a plurality of historical gas numerical control components at a plurality of pipeline network location information within the same gas region in a first vector database, and determine them as a reference battery replacement necessity degree and a reference component replacement necessity degree respectively; adjust the reference battery replacement necessity degree and the reference component replacement necessity degree based on the gas usage data of the gas region to determine the battery replacement threshold and the component replacement threshold. The adjustment manner may be such that the larger the total gas usage amount, the higher the replacement threshold. More descriptions regarding the first vector database may be found in FIG. 2.

In 522, the replacement parameter of the gas region is determined based on the replacement thresholds and the replacement necessity degree.

In some embodiments, the processor may determine the replacement parameter of the gas region based on the replacement thresholds and the replacement necessity degree in various ways. For example, for each gas region, the processor may compare the replacement threshold and the replacement necessity degree of each gas numerical control component within that gas region; in response to the battery replacement necessity degree of the gas numerical control component being greater than or equal to the battery replacement threshold and the component replacement necessity degree being less than the component replacement threshold, determine that the gas numerical control component is the component to be replaced, wherein a replacement manner is battery replacement; in response to the component replacement necessity degree of the gas numerical control component being greater than or equal to the component replacement threshold, determine that the gas numerical control component is the component to be replaced, wherein the replacement manner is overall component replacement.

In some embodiments of the present disclosure, by using the clustering algorithm, the plurality of gas regions of the gas pipeline network can be accurately divided, thereby determining the replacement thresholds corresponding to the gas numerical control components of different numerical control component types in different gas regions. This facilitates precise determination of the replacement parameters for each gas region, enables faster and more accurate determination of components to be replaced and the replacement manner, thus ensuring that gas data within the gas region is accurately and sufficiently monitored.

In some embodiments of the present disclosure, based on the pipeline network location information and the replacement necessity degree of the gas numerical control component in the gas pipeline network, the replacement parameters for the plurality gas regions of the gas pipeline network can be accurately determined regionally, which is conducive to more precise replacement of the gas numerical control components.

In some embodiments, the processor may determine one or more qualified components based on the replacement parameter; in response to receiving the replacement completion message, determine a synchronization collection parameter of the gas numerical control component and issue the synchronization collection parameter to the gas numerical control component; determine the collection confidence level of the gas numerical control component based on the gas monitoring data collected by the gas numerical control component according to the synchronization collection parameter; and perform validation on the collection confidence level.

More descriptions regarding the replacement parameter, the replacement completion message, the gas numerical control component, the gas monitoring data, and the collection confidence level may be found in FIGS. 2-5.

The qualified component refers to a gas numerical control component that does not need to be replaced. In some embodiments, the gas numerical control components before replacement include the qualified components and the components to be replaced; the gas numerical control components after replacement include the qualified components and the replaced components.

In some embodiments, the processor may determine the components to be replaced based on the replacement parameters, and determine other gas numerical control components, excluding the components to be replaced, as the qualified components.

The synchronization collection parameter refers to a unified collection parameter of the gas numerical control components after replacement, that is, a unified collection parameter for the qualified components and the replaced components.

In some embodiments, the synchronization collection parameter may include a synchronization collection period and a synchronization collection frequency. The synchronization collection time refers to a unified time period for data collection by the qualified components and the replaced components. The synchronization collection frequency refers to a unified frequency for data collection by the qualified components and the replaced components.

In some embodiments, in response to receiving the replacement completion message, the processor may determine a preset synchronization collection parameter as the synchronization collection parameter of the gas numerical control component. The preset synchronization collection parameter may be a manually set parameter or an average value of historical synchronization collection parameters determined by statistical calculation performed by the processor.

In some embodiments, the processor may issue the synchronization collection parameter to the gas numerical control component, and the gas numerical control component collects the gas monitoring data according to the synchronization collection parameter. The gas numerical control component includes the replaced component and the qualified component.

In some embodiments, the processor may determine the collection confidence level of the gas numerical control component based on the gas monitoring data collected by the gas numerical control component according to the synchronization collection parameter. For the process of determining the collection confidence level based on the gas monitoring data, please refer to the relevant description above, which will not be repeated here.

In some embodiments, the processor may perform validation on the collection confidence level in various ways. For example, in response to the collection confidence level being greater than a preset confidence level threshold, the processor may determine that the validation is successful; in response to the collection confidence level not being greater than the preset confidence level threshold, the processor may determine that the validation fails, then perform maintenance inspection on the gas numerical control component, and if it is determined that the gas numerical control component has a problem, the processor may perform replacement again.

In some embodiments of the present disclosure, the determining the collection confidence level of the gas numerical control component based on the gas monitoring data collected by the gas numerical control component according to the synchronization collection parameter, and performing validation on the collection confidence level can further ensure the reliability of the collection confidence level, thereby ensuring the accuracy of replacing the gas numerical control component and ensuring the normal operation of the gas pipeline.

In some embodiments, in response to completing the validation of the collection confidence level, the processor may determine a low-battery component among the qualified components; reduce a data collection parameter of the low-battery component, and increase the data collection parameter of the replaced component to maintain that a total collection amount satisfies a preset requirement.

More descriptions regarding the collection confidence level, the qualified component, the replaced component, and the data collection parameter may be found in the related descriptions above.

The low-battery component refers to a qualified component with a low battery level.

In some embodiments, the processor may determine the qualified component with a remaining battery level below a preset power value as the low-battery component.

The preset power value refers to a preset minimum battery level at which the gas numerical control component can continue to operate normally. The preset power value may be set by the processor by default or preset manually based on experience, such as 60% of full battery capacity.

The total collection amount refers to a total amount of data collected by all gas numerical control components for the gas monitoring data.

In some embodiments, the preset requirement may be set by the processor by default or preset manually based on experience in advance. For example, the preset requirement may be that a change amount of the total collection amount does not exceed a preset change threshold. The preset change threshold may also be set by the processor by default or preset manually based on experience in advance.

In some embodiments, the processor may maintain that the total collection amount satisfies the preset requirement by reducing the data collection parameter of the low-battery component and increasing the data collection parameter of the replaced component.

In some embodiments of the present disclosure, by determining the low-battery component among the qualified components, reducing the data collection parameter of the low-battery component, and increasing the data collection parameter of the replaced component to maintain that the total collection amount satisfies the preset requirement, it is possible to ensure sufficient gas monitoring data is collected while maximizing the working duration of the gas numerical control components, thereby reducing the replacement cost of the gas numerical control components while ensuring the normal operation of the gas pipeline network.

One or more embodiments of the present disclosure provide a computer-readable storage medium, wherein the computer-readable storage medium stores computer instructions. When read and executed by a computer, the computer executes the method for safely replacing the gas pipeline network component based on the supervision network.

The embodiments in the present disclosure are for illustration and description only and do not limit the scope of application of the present disclosure. For those skilled in the art, various modifications and changes that may be made under the guidance of the present disclosure are still within the scope of the present disclosure.

Certain features, structures, or characteristics in one or more embodiments of the present disclosure may be appropriately combined.

Some embodiments use numbers to describe ingredients, attribute quantities. It should be understood that such numbers used to describe embodiments are, in some examples, modified with the modifiers “approximately,” “about,” or “substantially.” Unless otherwise stated, “approximately,” “about,” or “substantially” indicates that the stated number allows for a variation of ±20%. Accordingly, in some embodiments, the numerical parameters used in the specification and claims are approximations that may vary depending on the characteristics required for individual embodiments. In some embodiments, numerical parameters should take into account the specified significant digits and apply the way of retaining digits generally. Although the numerical ranges and parameters used to confirm the breadth of their ranges in some embodiments of the present disclosure are approximations, in specific embodiments, the setting of such values is as precise as feasible within the possible range.

If there is any inconsistency or conflict between the descriptions, definitions, and/or use of terms in the materials cited in the present disclosure and the content described in the present disclosure, the descriptions, definitions, and/or use of terms in the present disclosure shall prevail.

What is claimed is:

1. A method for safely replacing a gas pipeline network component based on a supervision network, wherein the method is implemented by an internet of things (IoT) system for safely replacing the gas pipeline network component based on the supervision network, the IoT system comprising a government safety supervision service platform, a government safety supervision management platform, a government safety supervision sensing network platform, a government safety supervision object platform, a gas company sensing network platform, a smart gas equipment object platform, a citizen user platform, a gas user platform, and a gas user service platform, wherein:
the government safety supervision object platform includes a gas company management platform, the smart gas equipment object platform includes a gas supply control device, the gas user platform includes a maintenance user sub-platform and a gas user sub-platform, and the gas user service platform includes a maintenance service sub-platform, wherein
the method is executed by the gas company management platform and comprises:
acquiring a component feature of a gas numerical control component based on a data center of the gas company management platform, the component feature comprising at least one of a data processing feature, a working feature, and an environmental feature, the component feature being acquired by the smart gas equipment object platform, uploaded to the gas company management platform via the gas company sensing network platform, and stored in a data partition of the data center corresponding to the gas numerical control component;
determining a replacement necessity degree of the gas numerical control component based on the component feature;
determining a replacement parameter based on the replacement necessity degree;
generating a replacement task instruction and a gas shutoff parameter based on the replacement parameter, transmitting the replacement task instruction to the maintenance user sub-platform via the maintenance service sub-platform, and transmitting the gas shutoff parameter to the government safety supervision management platform via the government safety supervision sensing network platform;
in response to receiving a shutoff parameter confirmation instruction from the government safety supervision management platform, generating a gas shutoff instruction, and transmitting the gas shutoff instruction to the gas supply control device via the gas company sensing network platform to control an open/closed state of the gas supply control device;
generating gas shutoff reminder information and transmitting the gas shutoff reminder information to the gas user sub-platform and the citizen user platform; and
in response to receiving a replacement completion message issued by the maintenance user sub-platform, determining a replaced component and updating the data partition corresponding to the replaced component.

2. The method of claim 1, wherein
the working feature includes a first working feature and a second working feature, the first working feature is a working feature of the gas numerical control component during a first time period, the second working feature is a working feature of the gas numerical control component during a second time period, the first time period is a time period from a historical time point of a last battery replacement to a current time point, and the second time period is a time period from a historical time point of a last component replacement to the current time point;
the environmental feature includes a first environmental feature and a second environmental feature, the first environmental feature is an environmental feature of the gas numerical control component during the first time period, and the second environmental feature is an environmental feature of the gas numerical control component during the second time period;
the replacement necessity degree includes a battery replacement necessity degree and a component replacement necessity degree; and
the determining the replacement necessity degree of the gas numerical control component based on the component feature includes:
determining a remaining battery level of the gas numerical control component based on an initial battery level, the data processing feature, the first working feature, and the first environmental feature of the gas numerical control component;
determining the battery replacement necessity degree based on the remaining battery level;
determining a component wear degree of the gas numerical control component based on the second working feature, the second environmental feature, and an average gas feature; and
determining the component replacement necessity degree based on the component wear degree.

3. The method of claim 2, wherein the determining the component wear degree of the gas numerical control component based on the second working feature, the second environmental feature, and the average gas feature includes:
determining a wear coefficient of the gas numerical control component based on the second working feature, the second environmental feature, the average gas feature, and reference usage data; and
determining the component wear degree based on a component usage duration, a design service life, and the wear coefficient of the gas numerical control component.

4. The method of claim 2, further comprising:
acquiring gas monitoring data collected by the gas numerical control component from the data center;
determining a plurality of collection confidence levels of the gas numerical control component at a plurality time points based on the gas monitoring data;
determining an operational stability level of the gas numerical control component based on the plurality of collection confidence levels corresponding to the gas numerical control component at the plurality of time points; and
adjusting the component replacement necessity degree based on the collection confidence level at the time point and the operational stability level.

5. The method of claim 4, wherein the determining the plurality of collection confidence levels of the gas numerical control component at the plurality of time points based on the gas monitoring data includes:
constructing a data association graph based on a plurality of gas numerical control components located on a same reference gas pipeline, reference gas monitoring data collected by the plurality of gas numerical control components, pipeline information between the plurality of gas numerical control components, and numerical control component types of the plurality of gas numerical control components; and
determining the collection confidence level based on the data association graph and the gas monitoring data using an evaluation model, the evaluation model being a machine learning model.

6. The method of claim 1, wherein the replacement parameter includes replacement sub-parameters of a plurality of gas regions within the gas pipeline network, and the determining the replacement parameter based on the replacement necessity degree includes:
determining the replacement sub-parameters of the plurality of gas regions within the gas pipeline network based on pipeline network location information of the gas numerical control component in the gas pipeline network and the replacement necessity degree.

7. The method of claim 6, wherein the determining the replacement sub-parameters of the plurality of gas regions within the gas pipeline network based on pipeline network location information of the gas numerical control component in the gas pipeline network and the replacement necessity degree includes:
determining the plurality of gas regions of the gas pipeline network through a clustering algorithm based on the pipeline network location information of the gas numerical control component in the gas pipeline network and gas usage data of a gas pipeline where the gas numerical control component is located;
for each gas region,
determining replacement thresholds for gas numerical control components of different numerical control component types within the gas region based on the gas usage data of the gas region; and
determining the replacement sub-parameter of the gas region based on the replacement thresholds and the replacement necessity degree.

8. The method of claim 6, further comprising:
determining one or more qualified components based on the replacement parameter;
in response to receiving the replacement completion message, determining a synchronization collection parameter of the gas numerical control component and issuing the synchronization collection parameter to the gas numerical control component;
determining a collection confidence level of the gas numerical control component based on the gas monitoring data collected by the gas numerical control component according to the synchronization collection parameter; and
performing validation on the collection confidence level.

9. The method of claim 8, further comprising:
in response to completing the validation of the collection confidence level, determining a low-battery component among the one or more qualified components; and
reducing a first data collection parameter of the low-battery component, and increasing a second data collection parameter of the replaced component to maintain that a total collection amount satisfies a preset requirement.

10. An internet of things (IoT) system for safely replacing a gas pipeline network component based on a supervision network, wherein the IoT system for safely replacing the gas pipeline network component based on the supervision network comprises:
a government safety supervision service platform,
a government safety supervision management platform,
a government safety supervision sensing network platform,
a government safety supervision object platform,
a gas company sensing network platform,
a smart gas equipment object platform,
a citizen user platform,
a gas user platform, and
a gas user service platform, wherein:
the government safety supervision object platform includes a gas company management platform, the smart gas equipment object platform includes a gas supply control device, the gas user platform includes a maintenance user sub-platform and a gas user sub-platform, and the gas user service platform includes a maintenance service sub-platform, wherein
the gas company management platform is configured to:
acquire a component feature of a gas numerical control component based on a data center of the gas company management platform, the component feature comprising at least one of a data processing feature, a working feature, and an environmental feature, the component feature being acquired by the smart gas equipment object platform, uploaded to the gas company management platform via the gas company sensing network platform, and stored in a data partition of the data center corresponding to the gas numerical control component;
determine a replacement necessity degree of the gas numerical control component based on the component feature;
determine a replacement parameter based on the replacement necessity degree;
generate a replacement task instruction and a gas shutoff parameter based on the replacement parameter, transmit the replacement task instruction to the maintenance user sub-platform via the maintenance service sub-platform, and transmit the gas shutoff parameter to the government safety supervision management platform via the government safety supervision sensing network platform;
in response to receiving a shutoff parameter confirmation instruction from the government safety supervision management platform, generate a gas shutoff instruction, and transmit the gas shutoff instruction to the gas supply control device via the gas company sensing network platform to control the open/closed state of the gas supply control device;
generate gas shutoff reminder information and transmit the gas shutoff reminder information to the gas user sub-platform and the citizen user platform; and
in response to receiving a replacement completion message issued by the maintenance user sub-platform, determine a replaced component and update the data partition corresponding to the replaced component.

11. The IoT system of claim 10, wherein:
the working feature includes a first working feature and a second working feature, the first working feature is the working feature of the gas numerical control component during a first time period, the second working feature is the working feature of the gas numerical control component during a second time period, the first time period is a time period from a historical time point of the last battery replacement to a current time point, and the second time period is a time period from a historical time point of the last component replacement to the current time point;
the environmental feature includes a first environmental feature and a second environmental feature, the first environmental feature is the environmental feature of the gas numerical control component during the first time period, and the second environmental feature is the environmental feature of the gas numerical control component during the second time period; and
the replacement necessity degree includes a battery replacement necessity degree and a component replacement necessity degree; wherein
the gas company management platform is further configured to:
determine a remaining battery level of the gas numerical control component based on an initial battery level, the data processing feature, the first working feature, and the first environmental feature of the gas numerical control component;
determine the battery replacement necessity degree based on the remaining battery level;
determine a component wear degree of the gas numerical control component based on the second working feature, the second environmental feature, and an average gas feature; and
determine the component replacement necessity degree based on the component wear degree.

12. The IoT system of claim 11, wherein the gas company management platform is further configured to:
determine a wear coefficient of the gas numerical control component based on the second working feature, the second environmental feature, the average gas feature, and reference usage data; and
determine the component wear degree based on a component usage duration, a design service life, and the wear coefficient of the gas numerical control component.

13. The IoT system of claim 11, wherein the gas company management platform is further configured to:
acquire gas monitoring data collected by the gas numerical control component from the data center;
determine a plurality of collection confidence levels of the gas numerical control component at a plurality time points based on the gas monitoring data;
determine an operational stability level of the gas numerical control component based on the plurality of collection confidence levels corresponding to the gas numerical control component at the plurality of time points; and
adjust the component replacement necessity degree based on the collection confidence level at the time point and the operational stability level.

14. The IoT system of claim 13, wherein the gas company management platform is further configured to:
construct a data association graph based on a plurality of gas numerical control components located on a same reference gas pipeline, reference gas monitoring data collected by the plurality of gas numerical control components, pipeline information between the plurality of gas numerical control components, and numerical control component types of the plurality of gas numerical control components; and
determine the collection confidence level based on the data association graph and the gas monitoring data using an evaluation model, the evaluation model being a machine learning model.

15. The IoT system of claim 10, wherein the replacement parameter includes replacement sub-parameters of a plurality of gas regions within the gas pipeline network, and the gas company management platform is further configured to:
determine the replacement sub-parameters of a plurality of gas regions within the gas pipeline network based on pipeline network location information of the gas numerical control component in the gas pipeline network and the replacement necessity degree.

16. The IoT system of claim 15, wherein the gas company management platform is further configured to:
determine the plurality of gas regions of the gas pipeline network through a clustering algorithm based on the pipeline network location information of the gas numerical control component in the gas pipeline network and gas usage data of a gas pipeline where the gas numerical control component is located;
for each gas region,
determine replacement thresholds for gas numerical control components of different numerical control component types within the gas region based on the gas usage data of the gas region; and
determine the replacement sub-parameter of the gas region based on the replacement thresholds and the replacement necessity degree.

17. The IoT system of claim 15, wherein the gas company management platform is further configured to:
determine one or more qualified components based on the replacement parameter;
in response to receiving the replacement completion message, determine a synchronization collection parameter of the gas numerical control component and issue the synchronization collection parameter to the gas numerical control component;
determine a collection confidence level of the gas numerical control component based on the gas monitoring data collected by the gas numerical control component according to the synchronization collection parameter; and
perform validation on the collection confidence level.

18. The IoT system of claim 17, wherein the gas company management platform is further configured to:
in response to completing the validation of the collection confidence level, determine a low-battery component among the one or more qualified components; and
reduce a first data collection parameter of the low-battery component, and increase a second data collection parameter of the replaced component to maintain that a total collection amount satisfies a preset requirement.

19. A computer-readable storage medium, wherein the computer-readable storage medium stores computer instructions, wherein when read and executed by a computer, the computer executes the method for safely replacing the gas pipeline network component based on the supervision network as recited in claim 1.
